(19) United States
(12) Patent Application Publication
(10) Pub. No.: US 2017/0089899 A1
(43) Pub. Date: Mar. 30, 2017

(54) MICROFLUIDIC CAPTURE AND DETECTION OF BIOLOGICAL ANALYTES

(71) Applicant: Cyclic Solutions, LLC, New Orleans, LA (US)

(72) Inventors: Kathryn Kundrod, Houston, TX (US); John Fraser, Arvada, CO (US)

(21) Appl. No.: 15/278,460

(22) Filed: Sep. 28, 2016

Related U.S. Application Data
(60) Provisional application No. 62/234,017, filed on Sep. 28, 2015.

(51) Int. Cl. G01N 33/569; G01N 27/327; G01N 27/416; B01L 3/00

(52) U.S. Cl. CPC G01N 33/56988; B01L 3/502715; B01L 3/502761; G01N 27/3277; G01N 27/416; B01L 2300/12; B01L 2300/0887; B01L 2300/0645; B01L 2200/0647; G01N 2333/16

(57) ABSTRACT

A microfluidic capture device is described that include a regular macroporous layer comprising a binding molecule, an inlet and outlet port fluidly connected to the regular macroporous layer and configured to add or remove a biological sample to the regular macroporous layer and positioned to allow flow of the biological sample through a flow region in the regular macroporous layer, a first and second electrode positioned on opposite sides of the flow region, and a substrate enclosing the regular macroporous layer, including top and bottom sides on opposite sides of the regular macroporous layer. Methods of using the device to qualitatively and/or quantitatively determine the amount of a biological analyte such as a virus particle in a biological sample obtained from a subject using cyclic voltammetry are also described.

### CONTINUING APPLICATION DATA

This application claims the benefit of U.S. Provisional Application Ser. No. 62/234,017, filed Sep. 28, 2015, the disclosure of which is incorporated by reference herein.

### TECHNICAL FIELD

This disclosure relates to systems and methods for detecting and quantifying biological material within a sample, and more particularly to systems and methods for quantitative viral sensing, including quantitative viral sensing employing electrochemical cyclic voltammetry.

### BACKGROUND

Viruses and other biological material and analytes are often blood borne. The amount of the analyte in the blood can indicate the presence of a disease, as well as the success of treatment and/or staging of the disease.

Testing of the quantity of a virus within a patient's blood is a useful proxy for the severity of the viral infection. The amount of the virus, that is the concentration of virus circulating in the blood, often referred to as the viral load, can be used to monitor viral infection, guide treatment, determine the effectiveness of treatment, and predict how a disease caused by the infection may progress. Measurement of viral load is of particular importance for the treatment of HIV infection. In conventional methods for determining HIV viral load, a whole blood sample is obtained from a patient by venipuncture. Cells are then removed from the sample by centrifugation to provide plasma, and the number of copies of HIV RNA per milliliter of plasma is determined, for example, by reverse-transcriptase polymerase chain reaction (RT-PCR), branched DNA (bDNA), or nucleic acid sequence-based amplification (NASBA) analysis. A high HIV viral load may indicate treatment failure, i.e. that the virus is replicating and the disease may progress more quickly. See International Publication WO 2014140641 A1. Given this, once a patient is diagnosed as HIV-positive and undergoing antiretroviral therapy, tests of viral load may be performed routinely to monitor disease progression and ensure treatment effectiveness. Although useful, viral load testing can be labor intensive, expensive and time consuming. It often takes upwards of two weeks to receive a patient's viral load results from central facilities, making it difficult for doctors to make treatment decisions or adjust medication in the case of drug resistance. This can be a significant burden on the patient, many of whom live in remote areas where it is difficult to meet with physicians, particularly for follow up visits about test results.

Currently, 35 million people in the world are living with HIV, the majority of whom reside in low- and middle-income countries. To diagnose, stage and monitor HIV infection and progression, viral load measurement is an imperative test. The World Health Organization (WHO) recommends viral load tests at least once per year for every person who begins antiretroviral therapy (ART) to stop the progression of an HIV infection. Conventionally, viral loads are measured using central laboratory-based tests, which require infrastructure, cold-chain transport, and trained personnel. To address the global pandemic of HIV, tests designed for use at the point of care that can be run with a portable setup, with a turn-around time of less than an hour, and require minimal training are urgently needed

### SUMMARY OF THE INVENTION

The inventors have developed a microfluidic solution for the capture and quantification of biological analytes such as viral particles. A novel porous membrane that has been proven effective in capturing HIV was transformed into a system that is capable of moving from sample to answer on-chip. They have created a technology that is appropriate for point-of-care applications. The invention includes incorporating a cyclic voltammetric system into microfluidic devices containing a porous membrane. This system is able to quantify viral loads of 1,000 copies per mL, which is the limit of detection required for point-of-care viral load technologies as recommended by the World Health Organization.

The device of the present invention is easier to operate, has a faster turnaround time, and is less expensive than nucleic acid amplification-based tests. This lab-on-a-chip design utilizes microfluidics for capture and quantification of whole HIV virions. The small size of microfluidic devices and the potential to automate assays make microfluidic technology appealing for point-of-care settings. Directly detecting whole particle virions instead of their molecular fingerprints, minimizes sample preparation procedure, resulting in a faster sample-to-answer timeframe.

The systems, methods and devices of this disclosure each have several innovative aspects, no single one of which is solely responsible for the desirable attributes disclosed herein.

Details of one or more implementations of the subject matter described in this disclosure are set forth in the accompanying drawings and the description below. Other features, aspects, and advantages will become apparent from the description, the drawings and the claims. Note that the relative dimensions of the following figures may not be drawn to scale.

### BRIEF DESCRIPTION OF THE FIGURES

The present invention may be more readily understood by reference to the following drawings wherein:

FIG. 1 provides a schematic representation of the microfluidic capture device as part of a system including tubing to facilitate flow of solution through the device and electrodes connected to a potentiostat to provide current to carry out cyclic voltammetry, and a computer to monitor the data generated by the potentiostat.

FIG. 2 provides an exploded view of the microfluidic capture device.

FIG. 3 provides a schematic representation of a method for fabricating a regular macroporous layer. A binary suspension of polystyrene and silica beads is deposited into a PDMS mold. The polystyrene is melted, and the silica is etched away, leaving behind a macroporous layer that can be incorporated into a device.

FIG. 4 provides an image showing the steps of aggregate building illustrated over a SEM image of the regular macroporous layer. First, a binding molecule (e.g., anti-gp120 antibody) is associated with the pores within the macroporous layer. At this stage, ions would be fully able to reach the electrodes. Second, a biological analyte (e.g., HIV) is captured by the antibody. Third, gold nanoparticles are used to functionalize the anti-gp120 bound to the captured HIV. Finally, silver is deposited around the bound gold nanoparticles. With each of these steps, ions are less and less able to reach the electrodes.

FIG. 5 provides a graph showing cyclic volammetric curves for two viral loads. Peak currents are shown at the rightmost end of the top 2 curves.

FIG. 6 provides a graph showing the standardized peak current versus the viral load for simulated HIV (biotinylated polystyrene beads 100 nm in diameter).

FIG. 7 provides a graph showing the standardized peak current versus the viral load for HIV pseudo-virus.

FIG. 8 provides a graph showing differentiation between control viral load (0 copies/mL) and threshold viral load for adjusting treatment regimens (1,000 copies/mL). The Asterisk indicates a statistically significant difference (p=0.0244) between peak currents measured for each viral load. This demonstrates that the limit of detection is appropriate for point-of-care applications.

To illustrate the invention, several embodiments of the invention will now be described in more detail. Reference will be made to the drawings, which are summarized above. Skilled artisans will recognize the embodiments provided herein have many useful alternatives that fall within the scope of the invention.

### DETAILED DESCRIPTION OF THE INVENTION

This disclosure provides systems, methods and apparatus that integrate sample preparation and whole particle viral detection into a sample-to-answer system that attains clinically relevant limits of detection. Quantitative sensing of biological analytes is achieved by electrochemical cyclic voltammetry. While it has been reported as a sensitive method to detect biomolecules, such as proteins and nucleic acids, no groups have used cyclic voltammetry to detect whole virus. Electrochemical detection is traditionally used to measure the strength of a chemical reaction in aqueous systems of varying ion compositions. Cyclic voltammetry is a form of electrochemical detection that has potential for high sensitivity. In order to perform cyclic voltammetry for viral load detection, the inventors designed a microfluidic device that incorporates electrodes in a novel way. Device design, signal amplification, and sensitive cyclic voltammetric detection, among other things, are novel aspects of this invention.

### DEFINITIONS

Unless otherwise defined, all technical and scientific terms used herein have the same meaning as commonly understood by one of ordinary skill in the art to which this invention pertains. In case of conflict, the present specification, including definitions, will control.

The terminology as set forth herein is for description of the embodiments only and should not be construed as limiting of the invention as a whole. Unless otherwise specified, “a,” “an,” “the,” and “at least one” are used interchangeably. Furthermore, as used in the description of the invention and the appended claims, the singular forms “a”, “an”, and “the” are inclusive of their plural forms, unless contraindicated by the context surrounding such.

The terms “comprising” and variations thereof do not have a limiting meaning where these terms appear in the description and claims.

Also herein, the recitations of numerical ranges by endpoints include all numbers subsumed within that range (e.g., 1 to 5 includes 1, 1.5, 2, 2.75, 3, 3.80, 4, 5, etc.).

As used in this invention, the term “epitope” means any antigenic determinant on an antigen to which the antibody binds. Epitopic determinants usually consist of chemically active surface groupings of molecules such as amino acids or sugar side chains and usually have specific three dimensional structural characteristics, as well as specific charge characteristics. Epitopes of the invention can be present, for example, on cell surface receptors.

The devices and methods described herein recognize that microfluidic technology offers the ability to analyze small sample volumes, encouraging the development of point-of-care systems for bioanalyte detection (e.g., viral diagnostics). A microfluidic viral load analyzer may separate, among other things, HIV virions from plasma and quantify the targets. The small size of virions limits the use of traditional, flat-bed, immunoaffinity microfluidic devices. Thus, in certain implementations, the systems and methods described herein employ a regular macroporous layer to isolate HIV virions from a solution.

### Microfluidic Capture Device

One aspect of the present invention provides a microfluidic capture device. The device includes a regular macroporous layer comprising a binding molecule, an inlet and outlet port fluidly connected to the regular macroporous layer and configured to add or remove a biological sample to the regular macroporous layer and positioned to allow flow of the biological sample through a flow region in the regular macroporous layer, a first and second electrode positioned on opposite sides of the flow region, and a substrate enclosing the regular macroporous layer, including top and bottom sides on opposite sides of the macroporous layer.

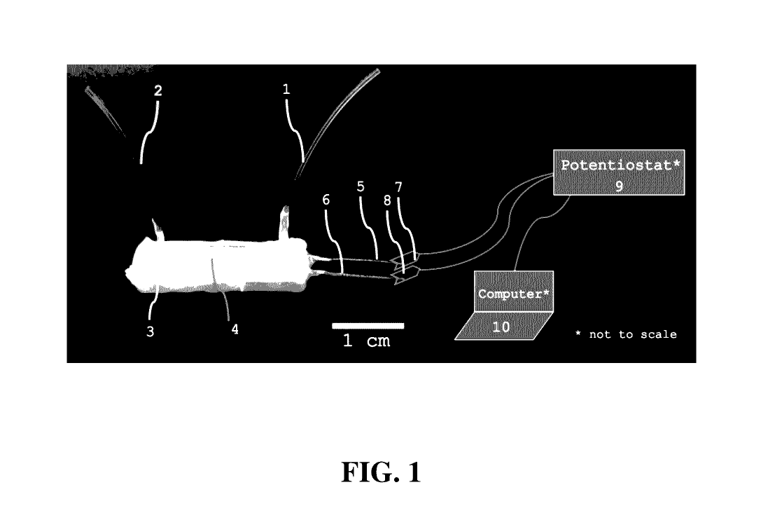

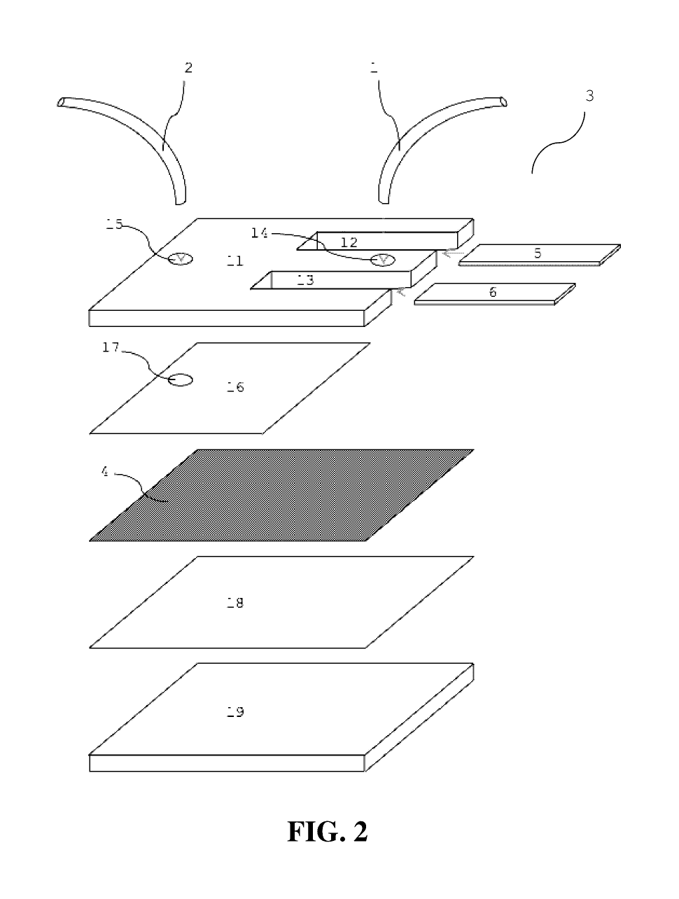

FIG. 1 depicts a microfluidic capture device able to capture and quantify an analyte of interest. The device shown in FIG. 1 allows for capture and detection of whatever bioanalyte the incorporated binding molecule specifically binds to. An illustration of the device and associated instrumentation used to run analysis is provided in FIG. 1, with the scale bar corresponding to embodiments shown. The device is comprised of flexible inlet tubing (1) and outlet (2) tubing (e.g., Tygon® Microbore Tubing, Norton Performance Plastics) to allow for delivery of analyte solution to and from the device. The details of the device (3), including the regular macroporous layer (4) within the device (3), are shown in greater detail in FIG. 2. First and second leads (7) & (8) can be used to connect the first and second electrodes to a current source such as a potentiostat (9) (e.g., from Gamry Instruments™). The potentiostat is then connected to a computer (10), though other control sources could be used.

An exploded view of the microfluidic capture device (3) is shown in FIG. 2. A first electrode (5) and second electrode (6) are incorporated into the top section (11) of the device such that they make contact with the regular macroporous layer (4). The first component of the device casing is the top section (11) of the substrate. In the embodiment shown, the top section (11) includes a first channel (12) and a second channel (13) for receiving the electrodes. The top section (11) also includes an inlet port (14) and outlet port (15) that are fluidly connected to the regular macroporous layer (4) and configured to add or remove a biological sample to the regular macroporous layer (4) and positioned to allow flow of the biological sample through a flow region in the regular macroporous layer (4). In some embodiments, top section (11) is attached to the regular macroporous layer (4) using an adhesive layer (16) (e.g., double-sided tape) that includes an adhesive layer hole (17) that aligns with the outlet port (15). The adhesive layer (16) only covers a portion of the regular macroporous layer (4), and is positioned such that it does not interfere with passage of the biological sample through the inlet port (14) to the regular macroporous layer (4), or between the electrodes and the regular macroporous layer. The regular macroporous layer (4) can be attached to a bottom section (19) of the substrate using a second adhesive layer (18). The second adhesive layer (18) can be formed with a suitable adhesive such as epoxy (PC-Products®), and typically has dimensions corresponding to the regular macroporous layer (4).

### Regular Macroporous Layer

The microfluidic capture device includes a regular macroporous layer. The regular macroporous layer includes a binding molecule, or more typically a plurality of binding molecules, and is fluidly connected through the inlet and outlet ports to the inlet and outlet tubing to allow flow of the biological sample into the regular macroporous layer and through a flow region in the regular macroporous layer. The portion of the regular macroporous layer through which the biological sample flows from where it enters at the input port to where it leaves through the outlet port is referred to herein as the flow region. The size of the flow region can vary depending on the size of the input and outlet ports, and the amount of diffusion into the regular macroporous layer that occurs, which will vary depending on a variety of factors, such as the pore size within the regular macroporous layer and the rate of flow of the biological sample.

The regular macroporous layer can be formed from any suitable material, but typically the regular macroporous layer is formed using a polymer. For example, in some embodiments, the macroporous layer comprises polystyrene. The regular macroporous layer is porous to allow flow of the biological sample through the layer. In addition, the macroporous layer includes a regular structure that facilitates flow through the layer and can be blocked with biological analytes having a particular diameter. The regular structure is the result of the existence of uniform and repeatable pores within the layer, which include interconnections between the pores (referred to herein as pore interconnections) that allow liquid flow through a series of interconnected pores. Pore interconnections represents gaps that exist between adjacent pores. The regular macroporous layer also includes pores that are larger than those typically found in a polymer matrix, hence use of the term “macroporous.”

The size of the pores and pore interconnections can vary from one embodiment of the invention to another, depending on the biological analyte of interest. The pore interconnections should be larger than the biological analyte to allow flow of the biological analyte through the regular macroporous layer, and the pores are typically substantially larger than the pore interconnections. Essentially, the pore intereconnections are the bottlenecks limiting flow through the regular macroporous layer. The pore interconnections range in size from about 50 nm to about 10 μm, with some embodiments including pore interconnections having a size of about 100 nm, 200 nm, 500 nm, 1 μm, and 10 μm. The pores themselves are much larger, and range in size from about 0.5 μm to about 50 μm, with some embodiments including pores having a diameter from about 0.5 μM to 10 μM.

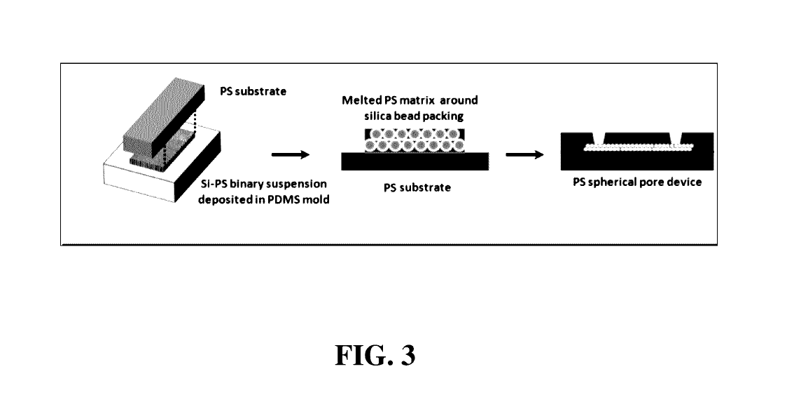

A variety of methods are known to those skilled in the art for creating layers having a regular macroporous structure. See for example Surawathanawises et al., Analyst, 141(5):1669-77 (2016), the disclosure of which is incorporated herein by reference. In some embodiments, the regular macroporous structure is formed using binary convective deposition. For a description of use of the binary convective deposition process, see Weldon et al., ACS Appl Mater Interfaces, 4(9):4532-40 (2012), the disclosure of which is incorporated herein by reference. A method of fabricating macroporous membranes is shown in FIG. 3. Convective deposition is used to create crystalline thin films containing two types of particles; nanoparticles that remain as the polymeric membrane and larger microspheres that are sacrificed to form cavities and macropores within the layer. For example, in one embodiment of this method, a thin film consisting of ordered SiO₂ micropheres and polystyrene nanoparticles are co-deposited with highly uniform local microstructure, long-range morphology, and film thickness. After melting the polystyrene particles and etching away SiO₂, a continuous polystyrene porous phase is obtained. When using this method, the regular macroporous layer comprises a regular array of microspherical voids.

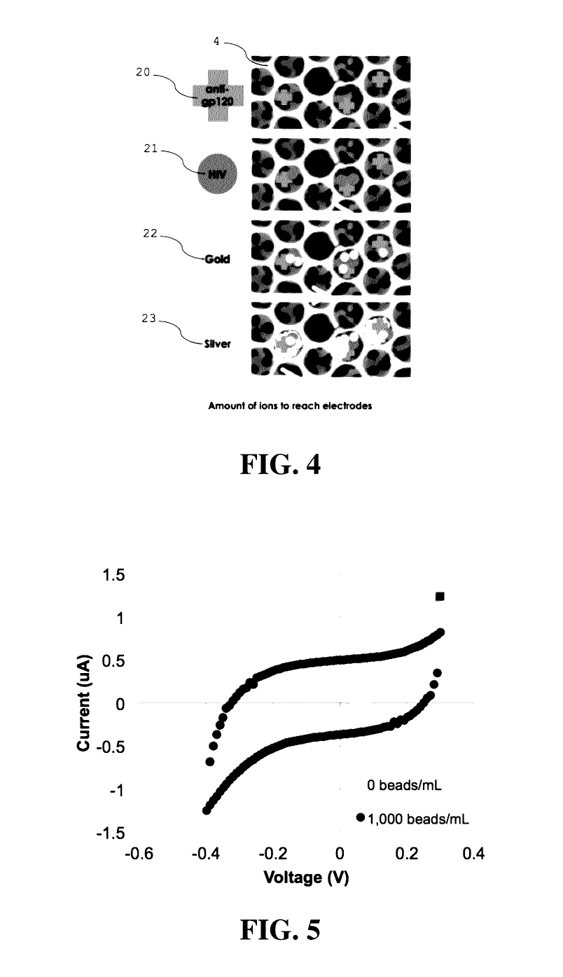

A method through which one embodiment of the regular macroporous layer can be used in accordance with the invention is shown in FIG. 4. FIG. 4 shows a method of using the regular macroporous layer to detect human immunodeficiency virus (HIV) using anti-gp120 as the binding molecule. The figure shows how HIV particles were captured within the regular macroporous layer (4) using anti-gp120 antibody, and then building aggregates around the HIV particles, forming blockages within regular macroporous layer. The blockages act as resistance, preventing ion flow through the device during cyclic voltammetry. More specifically, the membrane is functionalized with a binding molecule (e.g., anti-gp120) (20). Next, HIV (21) is captured by the binding molecule. To enhance blockage, gold nanoparticles (22) are bound to the captured HIV, and finally, silver (23) is deposited around the gold nanoparticles. As can be seen in the figure, the pores of the regular macroporous layer show increasing levels of blockage as the HIV is bound, and then gold and silver are deposited.

### Biological Analytes

The present invention provides a microfluidic capture device and methods of using the device to detect the presence and/or amount of a wide variety of biological analytes. Biological analytes, as used herein, refers to molecules associated with microorganisms, and in some embodiments infective microorganisms. The biological analytes can be the microorganism itself, a part of the microorganism, or a factor secreted by the microorganism. Examples of microorganisms include viruses, bacteria, fungi, and protozoa. The diameter of the biological analyte can vary from 1 nanometer to 10 micrometers, while in other embodiments the diameter of the biological analyte is from about 10 nanometers to about 1 micrometer. For example, hepatitis B virus particles have a diameter of 42 nanometers, ebola virus particles have a diameter of 80 nanometers, and bacterial cells typically have a diameter of about 1 micrometer. In some embodiments, the biological analyte is a protein or an antigenic fragment of a protein that can be used to help detect the microorganism as a result of binding to an antibody, antibody, fragment, or other suitable ligand. However, in other embodiments, the biological analyte can be other detectable material, such as polynucleotides (e.g., DNA or RNA) that can be bound using aptamers.

The microfluidic capture device and methods for its use have been validated for HIV viral load quantification, but can readily be applied to the detection of other analytes of various size and specific antibody affinity. The validation of the device using HIV demonstrates that the membrane can capture other biological analytes such as spherical biomolecules (e.g., enveloped virus strains). Examples of spherical viruses that could potentially be detected are rhinovirus, Dengue fever virus, Coronavirus, and Herpes simplex virus. The device has the potential to detect molecules of other geometries such as the rod shaped Ebola virus, Hepatitis B virus, Influenza A virus, and Bacillus anthracia, the bacterial cause of anthrax.

### Binding Molecules

The device includes a binding molecule that specifically binds to a biological analyte. A variety of binding molecules are known to those skilled in the art, such as antibodies, antibody fragments, binding ligands, and aptamers.

In some embodiments, the binding molecule is an antibody. Antibodies include polyclonal and monoclonal antibodies, as well as antibody fragments that contain the relevant antigen binding domain of the antibodies. The term “antibody” as used herein refers to immunoglobulin molecules or other molecules which comprise at least one antigen-binding domain. The term “antibody” as used herein is intended to include whole antibodies, monoclonal antibodies, polyclonal antibodies, chimeric antibodies, humanized antibodies, primatized antibodies, multi-specific antibodies, single chain antibodies, epitope-binding fragments, e.g., Fab, Fab′ and F(ab′)₂, Fd, Fvs, single-chain Fvs (scFv), disulfide-linked Fvs (sdFv), fragments comprising either a VL or VH domain, and totally synthetic and recombinant antibodies. The antibodies can be of any type (e.g., IgG, IgE, IgM, IgD, IgA, and IgY), class (e.g., IgG1, IgG2, IgG3, IgG4, IgA1 and IgA2) or subclass of immunoglobulin molecule.

Monoclonal antibodies may be produced in animals such as mice and rats by immunization. B cells can be isolated from the immunized animal, for example from the spleen. The isolated B cells can be fused, for example with a myeloma cell line, to produce hybridomas that can be maintained indefinitely in in vitro cultures. These hybridomas can be isolated by dilution (single cell cloning) and grown into colonies. Individual colonies can be screened for the production of antibodies of uniform affinity and specificity. Hybridoma cells may be grown in tissue culture and antibodies may be isolated from the culture medium. Hybridoma cells may also be injected into an animal, such as a mouse, to form tumors in vivo (such as peritoneal tumors) that produce antibodies that can be harvested as intraperitoneal fluid (ascites). The lytic complement activity of serum may be optionally inactivated, for example by heating.

Biological analytes (e.g., polypeptides or effective fragments thereof) may be used to generate antibodies. One skilled in the art will recognize that the amount of polypeptides used for immunization will vary based on a number of factors, including the animal which is immunized, the antigenicity of the polypeptide selected, and the site of injection. The polypeptides used as an immunogen may be modified as appropriate or administered in an adjuvant in order to increase the peptide antigenicity. In some embodiments, polypeptides, peptides, haptens, and small compounds may be conjugated to a carrier protein to elicit an immune response or may be administered with and adjuvant, e.g. incomplete Freund's adjuvant.

Protocols for generating antibodies, including preparing immunogens, immunization of animals, and collection of antiserum may be found in Antibodies: A Laboratory Manual, E. Harlow and D. Lane, ed., Cold Spring Harbor Laboratory (Cold Spring Harbor, N.Y., 1988) pp. 55-120 and A. M. Campbell, Monoclonal Antibody Technology: Laboratory Techniques in Biochemistry and Molecular Biology, Elsevier Science Publishers, Amsterdam, The Netherlands (1984).

The term “antibody fragment” as used herein is intended to include any appropriate antibody fragment which comprises an antigen-binding domain that displays antigen binding function. Antibodies can be fragmented using conventional techniques. For example, F(ab′)₂ fragments can be generated by treating the antibody with pepsin. The resulting F(ab′)₂ fragment can be treated to reduce disulfide bridges to produce Fab′ fragments. Papain digestion can lead to the formation of Fab fragments. Fab, Fab′ and F(ab′)₂, scFv, Fv, dsFv, Fd, dAbs, T and Abs, ds-scFv, dimers, minibodies, diabodies, bispecific antibody fragments and other fragments can also be synthesized by recombinant techniques or can be chemically synthesized. Techniques for producing antibody fragments are well known and described in the art. Antibody fragments, including single-chain antibodies, may comprise the variable region(s) alone or in combination with the entirety or a portion of the following: hinge region, CH1, CH2, and CH3 domains.

Antibodies are designed for specific binding, as a result of the affinity of complementary determining region of the antibody for the epitope of the biological analyte. An antibody “specifically binds” when the antibody preferentially binds a target structure, or subunit thereof, but binds to a substantially lesser degree or does not bind to a biological molecule that is not a target structure. In some embodiments, the antibody specifically binds to a virus particle such as the human immunodeficiency virus. An antibody specific for gp120 can be used to specifically bind to HIV particles, and can be an antibody or antibody fragment capable of binding to gp120 with a specific affinity of between 10⁻⁸ M and 10⁻¹¹ M. In some embodiments, an antibody or antibody fragment binds to gp120 with a specific affinity of greater than 10⁻⁷M, 10⁻⁸M, 10⁻⁹M, 10⁻¹⁰M, or 10⁻¹¹M, between 10⁻⁸M-10⁻¹¹M, 10⁻⁹M-10⁻¹⁰M, and 10⁻¹⁹M-10⁻¹¹M. In a preferred aspect, specific activity is measured using a competitive binding assay as set forth in Ausubel FM, (1994). Current Protocols in Molecular Biology. Chichester: John Wiley and Sons (“Ausubel”), which is incorporated herein by reference.

In some embodiments, the binding molecule is an aptamer. An aptamer is a nucleic acid that binds with high specificity and affinity to a particular target molecule or cell structure, through interactions other than Watson-Crick base pairing. Aptamer functioning is unrelated to the nucleotide sequence itself, but rather is based on the secondary/tertiary structure formed, and are therefore best considered as non-coding sequences. Aptamers of the present invention may be single stranded RNA, DNA, a modified nucleic acid, or a mixture thereof. The aptamers can also be in a linear or circular form. Accordingly, in some embodiments, the aptamers are single stranded DNA, while in other embodiments they are single stranded RNA.

The length of the aptamer of the present invention is not particularly limited, and can usually be about 10 to about 200 nucleotides, and can be, for example, about 100 nucleotides or less, about 50 nucleotides or less, about 40 nucleotides or less, or about 35 nucleotides or less. When the total number of nucleotides present in the aptamer is smaller, chemical synthesis and mass-production will be easier and less costly. In addition, in almost all known cases, the various structural motifs that are involved in the non-Watson-Crick type of interactions involved in aptamer binding, such as hairpin loops, symmetric and asymmetric bulges, and pseudoknots, can be formed in nucleic acid sequences of 30 nucleotides or less.

The aptamers of the invention are capable of specifically binding to biological analytes. Specific binding refers to binding which discriminates between the selected target and other potential targets, and binds with substantial affinity to the selected target. Substantial affinity represents an aptamer having a binding dissociation constant of at least about 10⁻⁸ mol/m³, but in other embodiments, the aptamers can have a binding dissociation constant of at least about 10⁻⁹ mol/m³, about 10⁻¹⁰ mol/m³, about 10⁻¹¹ mol/m³, or at least about 10⁻¹² mol/m³.

Aptamers can include structural analogs of the original aptamer. Examples of structural analogs include aptamers modified at the 2′-position hydroxyl group of pyrimidine or purine nucleotides with a hydrogen atom, halogen, or an —O-alkyl group. Wild-type RNA and DNA aptamers are not as stable as would be preferred because of their susceptibility to degradation by nucleases. Resistance to nuclease degradation can be greatly increased by the incorporation of modifying groups at the 2′-position. Examples of other modifications of aptamer nucleotides include 5-position pyrimidine modifications, 8-position purine modifications, modifications at exocyclic amines, substitution of 4-thiouridine, substitution of 5-bromo or 5-iodo-uracil; backbone modifications, and phosphorothioate or alkyl phosphate modifications.

### Electrodes

The microfluidic devices includes a first and second electrode positioned on opposite sides of the flow region so that ions can flow through the flow region between the electrodes when current is applied. The first and second electrode are placed in contact with the regular macroporous layer. In some embodiments, the top side of the substrate includes openings configured to retain the first and second electrodes on opposite sides of the flow region of the regular macroporous layer. However, the electrodes can also be placed on the bottom side of the substrate, or opposite sides of the substrate. In some embodiments, the openings have a shape corresponding to the shape of the electrodes to allow the substrate to better enclose the regular macroporous layer. It is preferable for the electrodes to be parallel to one another so that the distance between the electrodes is the same at all points along the electrodes. When the microfluidic capture device is used, the electrodes should be connected to a current source such as a potentiostat.

Incorporation of electrodes within the top substrate and contacting the top of the porous membrane turns, at least in part, on the geometry of the membrane. In one embodiment, the membrane consists of a structure of ordered pores. Particulates suspended within a fluid travel in random motion through this ordered network. Thus incorporating electrodes within the same plane allows for the least amount of variability of ionic travel from one electrode to the other, when the membrane is devoid of analyte (HIV). If electrodes were incorporated in different planes, one electrode on the roof of the membrane and the other electrode on the base of the membrane, the straight-line distance between the electrodes would increase and so would the average distance of random ionic travel. Thus electrodes are incorporated within the same plane in an effort to reduce path variability between devices and increase the consistency of the signal received. The first and second electrodes should be spaced apart sufficiently to encompass most of the flow region. The surface area of the electrodes can vary substantially, depending on the size of the device. For example, in different embodiments, the surface area of each working electrode can be about 1, 2, 5, or 10 mm² or greater. The first electrode may be the working electrode in a two-electrode system, and the second electrode may be the electrode that maintains a constant potential and a passes current. In this embodiment, the potential applied by the working electrode may be alternated between an oxidizing and a reducing potential. In some embodiments of the invention, a reference electrode is also included in the device.

Electrode(s) are fabricated using the methods and materials known in the art. Non-limiting examples of electro-conductive material suitable for electrode construction on the substrate layer include Copper, Nickel, Tin, Gold, Platinum, Stainless Steel, and conductive inks such as carbon ink or Ag/AgCl ink. In some embodiments, the electrode(s) are thin sheets of metal that are placed in contact with the regular macroporous layer. In other embodiments, other methods of constructing the electrodes on the macroporous layer can be used. Non-limiting examples of constructing the electrodes on the substrate layer include metal deposition (such as sputtering and sputter deposition, vapor deposition, thermal spray coating, and ion beam techniques), electrodeposition coating, etching, and self-assembly.

### Substrate Enclosing the Regular Macroporous Layer

The microfluidic capture device preferably includes a substrate including a top and a bottom side positioned on opposite sides of the regular macroporous layer. In some embodiments, the substrate encloses the regular macroporous layer. The substrate serves to protect the regular macroporous layer, retain biological sample in the regular macroporous layer, and reinforced the structure of the microfluidic device. In some embodiments, the substrate also includes a perimeter region (i.e., sides) that connects the top and bottom substrate and seals the microfluidic device.

The substrate can be cut or formed to include openings. For example, the substrate can include two openings that function as the inlet and outlet ports. These openings should be on the same portion of the substrate that includes the electrodes, and is typically the top portion of the substrate. The inlet and outlet ports are small openings through which liquid sample can be placed into and leave from the device. In some embodiments, they are configured to be attached to inlet and outlet tubing. The substrate can also include two openings in which the first and second electrode are placed. These openings, or channels, should be included in the same portion of the substrate, and are typically included in the top portion of the substrate, and/or the same portion of the substrate including the inlet and outlet ports.

The substrate may be formed of any suitable material or combination of suitable materials. Suitable materials may include elastomers, such as polydimethylsiloxane (PDMS); plastics, such as acrylic, polystyrene, polypropylene, polycarbonate, polymethyl methacrylate, etc.; glass; ceramics; sol-gels; silicon and/or other metalloids; metals or metal oxides; etc.

The substrate for the microfluidic device may be fabricated by any suitable mechanism, based on the desired application for the system and on materials used in fabrication. In some embodiments, the substrate and its features can be fabricated using a water jet cutter. In other embodiments, one or more components may be molded, stamped, and/or embossed using a suitable mold. Such a mold may be formed of any suitable material by micromachining, etching, soft lithography, material deposition, cutting, and/or punching, among others. Alternatively, or in addition, components of a microfluidic system may be fabricated without a mold by etching, micromachining, cutting, punching, and/or material deposition.

Microfluidic components may be fabricated separately, joined, and further modified as appropriate. For example, when fabricated as distinct layers, microfluidic components may be bonded, generally face-to-face. These separate components may be surface-treated, for example, with reactive chemicals to modify surface chemistry, with particle binding agents, with reagents to facilitate analysis, and/or so on. Such surface-treatment may be localized to discrete portions of the surface or may be relatively nonlocalized. In some embodiments, separate layers may be fabricated and then punched and/or cut to produce additional structure. Such punching and/or cutting may be performed before and/or after distinct components have been joined.

In some embodiments, the microfluidic capture device includes an adhesive layer between the regular macroporous layer and the top and bottom sides of the substrate. The adhesive layer attached the substrate to the regular macroporous layer, and can also provide a fluid-impermeable layer to help retain the analyte in the regular macroporous layer.

In certain embodiments, the adhesive layer is an adhesive sheet or tape. Double-sided tape adheres to two adjacent layers and can bind to other components of the microfluidic capture device. Non-limiting examples of materials suitable for use in the adhesive layer include Scotch™ double-sided carpet tape, water-impermeable barriers include 3M Double Sided Tape, Tapeworks double sided tape, CR Laurence black double sided tape, 3M Scotch Foam Mounting double-sided tape, 3M Scotch double-sided tape (clear), QuickSeam splice tape, double sided seam tape, 3M exterior weather-resistant double-sided tape, CR Laurence CRL clear double-sided PVC tape, Pure Style Girlfriends Stay-Put Double Sided Fashion Tape, Duck Duck Double-sided Duct Tape, and Electriduct Double-Sided Tape. In some embodiments, use of double sided adhesive tape to attach the substrate to the regular macroporous layer is preferred, since double sided tape can easily be cut or otherwise fashioned to cover only a portion of the regular macroporous layer to allow contact between the electrodes and the regular macroporous layer.

As an alternative to double-sided tape, a heat-activated adhesive can be used to attach the substrate to the regular macroporous layer. For example, in some embodiments, an epoxy resin can be used to attach the substrate to the regular macroporous layer.

### Methods of Detecting a Biological Analyte

In another aspect, the present invention provides a method of detecting a biological analyte in a subject, comprising obtaining a biological sample from a subject, passing the biological sample through the flow region of a microfluidic capture device as described herein, passing a redox solution through the flow region of the microfluidic capture device, applying a cyclic voltage to the first electrode of the microfluidic capture device, measuring the current flow and/or area of the voltage-current curve through the microfluidic capture device, and comparing the current flow and/or voltage-current curve to a corresponding control value to determine if the biological analyte is present in the biological sample, wherein the binding molecule of the microfluidic capture device specifically binds to the biological analyte being evaluated.

Once fabricated, the regular macroporous layer is functionalized with a binding molecule that isolates the biological analyte from solution. In some embodiments, the method further includes the step of functionalizing the binding molecule with gold nanoparticles after passing the biological sample through the flow region of the microfluidic capture device. In some embodiments, the method further comprises depositing silver on the gold nanoparticles. Functionalization, as used herein, refers to attachment of the additional material to a previously placed material, thereby modifying the function of the previously placed material. For example, gold nanoparticles functionalize binding molecules by increasing their size upon binding and increasing the blockage within the pores. A Method of functionalizing an immunoassay using gold nanoparticles and silver solution is described by de la Escosura-Muñiz, A. and Merkoçi, A. See de la Escosura-Muñiz, A., Merkoçi, “A Nanochannel/Nanoparticle-Based Filtering and Sensing Platform for Direct Detection of a Cancer Biomarker in Blood,” Small, 5, 675-682, (2011).

The method includes obtaining a biological sample from a subject and passing the biological sample through the flow region of a microfluidic capture device. Biological samples include, but are not necessarily limited to bodily fluids such as urine and blood-related samples (e.g., whole blood, serum, plasma, and other blood-derived samples), urine, cerebral spinal fluid, bronchoalveolar lavage, and the like.

A biological sample may be fresh or stored (e.g. blood or blood fraction stored in a blood bank). Samples can be stored for varying amounts of time, such as being stored for an hour, a day, a week, a month, or more than a month. The biological sample may be a bodily fluid expressly obtained for use in the microfluidic device of this invention or a bodily fluid obtained for another purpose which can be subsampled for the assays of this invention. In some embodiments, it may be preferable to filter, centrifuge, or otherwise pre-treat the biological sample to remove impurities or other undesirable matter that could interfere with analysis of the biological sample.

Detection of the biological analyte is facilitated by contacting the electrode surface with a redox solution comprising redox mediator in an electrolyte solution. Contact with the redox solution occurs subsequent to binding of the biological analyte by the binding molecules. The redox mediator in solution participates in a redox reaction which elicits electron transfer to the electrode surface, thus creating a detectable electrical current in the electrode. A variety of redox solutions are known to those skilled in the art. In some embodiments, the redox solution is a K[Fe(CN)₆] solution.

The term subject generally refers to an animal such as a vertebrate or invertebrate animal. In some embodiments, the subject is a mammal, including, but not limited to, primates, including simians and humans, equines (e.g., horses), canines (e.g., dogs), felines, various domesticated livestock (e.g., ungulates, such as swine, pigs, goats, sheep, and the like), as well as domesticated pets and animals maintained in zoos. In some embodiments, the subject is a human subject.

In some embodiments, the changed electrical characteristics of the device may be used to quantify the level of analyte present. For example, in some embodiments, the method can be used to quantify the viral load. Quantification of the viral load can be carried out at the same time as virus detection, or it can be carried out as a separate step. For example, in some embodiments, the subject may have already been diagnosed as having a viral infection, and the method is used to determine the viral load of the subject.

### Cyclic Voltammetry

Following capture within the device, the biological analytes change an electrical characteristic of the device. These systems and processes allow for viral load quantification in a point-of-care setting. In other embodiments, alternate means for evaluating a change in the electrical characteristics of the device can be used. Other methods include impedance measurement, amperometry (measurement of electrical currents), biamperometry, stripping voltammetry, differential pulse voltammetry, coulometry, and potentiometry. In some embodiments, the analytes within the fluidic sample are detected by chronoamperometric method. Chronoamperometry is an electrochemical technique in which the potential of the working electrode is stepped, and the resulting current from faradic processes occurring at the electrode (caused by the potential step) is monitored as a function of time.

In some embodiments, the methods and device integrate sample preparation and analyte detection into a sample-to-answer system that attains clinically relevant limits of detection. Quantitative sensing can be achieved by electrochemical cyclic voltammetry. Cyclic voltammetry is a form of electrochemical detection that has potential for high sensitivity. To perform cyclic voltammetry for viral load detection, the systems and methods employ a microfluidic device that incorporates electrodes into the device. As analyte bind to the binding molecules, blockages begin to arise within the regular macroporous layer. These blockages may be used, as explained more fully below, to prevent ions from traveling to an electrode. This, in turn, can change the current value through the electrodes and these measurements can be used to quantify the viral load in the sample.

To perform cyclic voltammetry, a redox solution containing free ions is introduced to the microfluidic device through the inlet port. When voltage is applied cyclically to the first electrode, ions move through the regular macroporous layer to the second electrode. The voltage applied can vary in different embodiments of the invention. In some embodiments, the voltage is applied cyclically from about −1 V to about +1 V, while in other embodiments the voltage is applied from about −0.5 V to about +0.5 V, while in a yet further embodiment the voltage applied is from about −0.3 V to about +0.4 V. If no analyte (e.g., virus) is present in the regular macroporous layer, ions are able to move freely through the layer and easily reach the second electrode, and the resultant peak current value is high. However, when there are blockages within the regular macroporous layer due to captured analyte (e.g., virus), which prevent ions from reaching the electrode, peak current values are lower. This inverse relationship allows for the measurement of peak current values, which can be used, for example, to diagnose a patient's viral load. An example of cyclic voltammetric curves for two viral loads (0 copies/mL and 1,000 copies/mL) is shown in FIG. 5. Additionally, the area bounded between the forward and reverse voltage sweeps can also be used to analyze viral load.

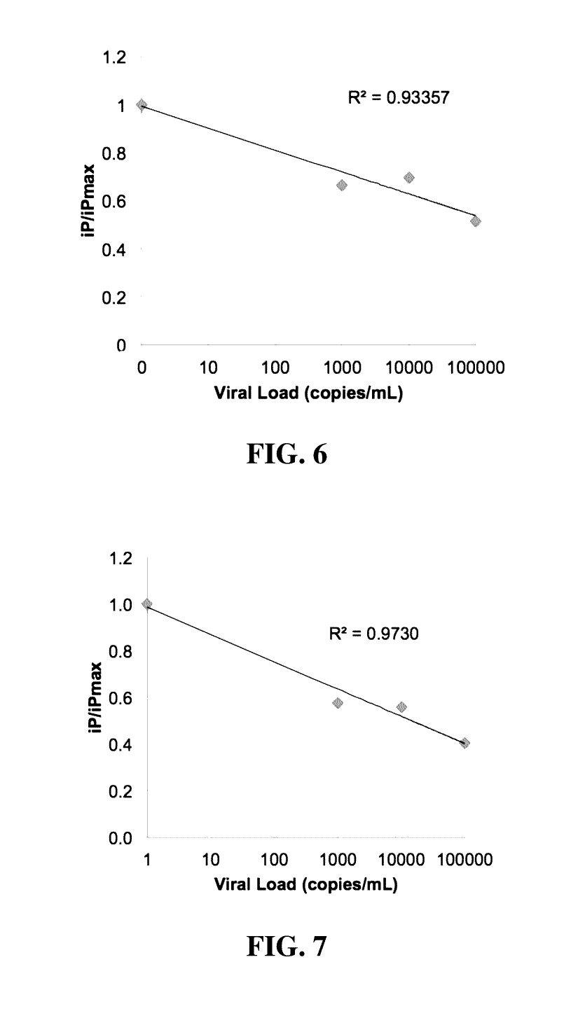

Due to biosafety considerations, evaluation of the device was first carried out using simulated HIV (biotinylated polystyrene beads, 100 nm in diameter). To simulate the capture reaction, the regular macroporous layer were coated with neutravidin, which has a strong binding affinity to biotin. To test the extent to which peak current and viral load have an inverse relationship, four concentrations of simulated virus (0, 1,000, 10,000, and 100,000 copies/mL) were flowed through devices, and peak currents obtained during cyclic voltammetry were recorded. The relationship proved linear with a fairly strong correlation (R²=0.93357), as can be seen in FIG. 6.

After proof of concept testing using simulated HIV (biotinylated polystyrene beads 100 nm in diameter), a second standard curve was generated using HIV pseudo-virus (n=16). HIV pseudo-virions carry functional viral envelopes and are structurally identical to that of active HIV virus, however they are absent of viral RNA and thus incapable of replication within a host cell. The standard curve obtained using HIV pseudo-virus is shown in FIG. 7. The standard curve was generated using four concentrations of pseudo-virus (0, 1,000, 10,000, and 100,000 virions/ml) (n=16), yielding an R² value of 0.9730. The relationship between the peak current and the viral load is inverse with a linear correlation, and can be used as a calibration curve to calculate viral load in an unknown sample using cyclic voltammetry measurement.

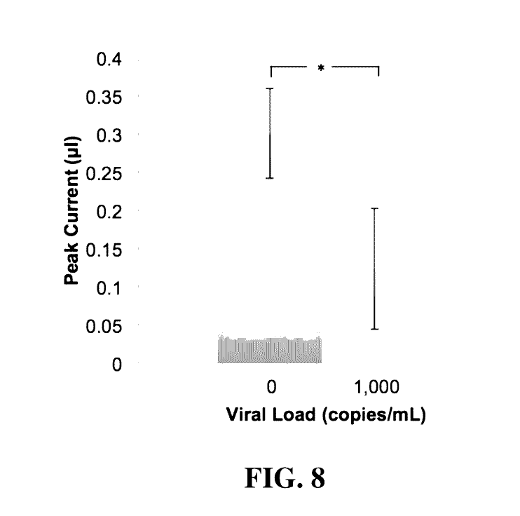

The World Health Organization defines the threshold of virological failure as a viral load of 1,000 copies/mL. Therefore, it is preferable for point-of-care viral load devices to have a limit of detection below or equal to 1,000 copies/mL. The embodiment of the microfluidic device tested by the inventors was able to differentiate between a control viral load of zero copies/mL and the threshold viral load of 1,000 copies/mL, supporting the clinical relevance of the design (FIG. 8).

Examples have been included to more clearly describe particular embodiments of the invention. However, there are a wide variety of other embodiments within the scope of the present invention, which should not be limited to the particular example provided herein.

### Example

### Example 1—Method of Fabricating the Regular Macroporous Layer

A methods of fabricating the regular macroporous layer is described. The polystyrene (PS) spherical pore devices were fabricated by templating close-packed silica beads. First, a polydimethyl siloxane (PDMS) open channel of 25 mm×8 mm×30 μm was fabricated using standard soft lithography. The PDMS surface was pretreated by a plasma gun to promote spreading of the suspension throughout the open channel. Then, 20 μL of a binary suspension of 1-μm silica and 100-nm PS in de-ionized water was pipetted into the PDMS channel. After drying, the silica beads self-assembled into ordered structures with the PS beads filling the interstitial space. Next, the PS nanobeads were melted at 240° C. for 10 min. The sample was glued to a PS flat sheet by epoxy glue and the PDMS mold was peeled off. Afterwards, silica beads were removed in 50% hydrofluoric acid and the device was rinsed in DI water. The porous matrix was attached to a flat piece of PS with drilled inlet and outlet by double-sided tape. Finally, the sides were sealed and the device was connected to tubing with epoxy glue. The porous region was 25 mm×8 mm×30 μm.

### Example II: Protocol for Fabrication a Microfluidic Capture Device

The steps of the protocol for fabricating a microfluidic capture device according to the present invention are described below, in numeric order.

i. A binary suspension is composed of 20% silica (SiO₂, diameter=1 μm) and 8% polystyrene (PS, diameter=0.2 μm).

ii. Using standard soft photolithography, a positive mold with dimensions 25 mm×8 mm×30 μm SU-8 photoresist is spun onto a silicon wafer.

iii. Poly(dimethyl siloxane) (PDMS) is poured over the mold and cured for two hours at 60° Celsius.

iv. The PDMS is cut in a rectangular shape enclosing the area of the mold.

v. The PDMS is placed on a glass slide with the negative mold facing up.

vi. The area of the negative mold is plasma oxidized for ten seconds.

vii. 20 μL of the binary suspension is deposited to fill the mold and spread evenly.

viii. After 30 minutes, the glass slide with PDMS and binary suspension is heated at 240° Celsius for 10 minutes, melting to the PS.

ix. After 20 minutes, a 25 mm×8 mm piece of poly(methyl methacrylate) (PMMA) sheet is epoxied to the binary suspension.

x. After 30 minutes, the binary suspension and bonded PMMA are peeled off of the PDMS.

xi. The PMMA with attached binary suspension is immersed in 50% hydrofluoric acid (HF) for 3 minutes, etching the silica out of the binary suspension and leaving behind a porous matrix.

xii. The PMMA with attached PS porous matrix is rinsed thoroughly with deionized water.

xiii. A 25 mm×8 mm piece of PMMA is water jet cut to have two holes on either nend (diameter=1 mm) and two channels (8 mm×1 mm) on one end to create a roof substrate.

xiv. Two 20 mm×1 mm stainless steel electrodes are cut by water jet.

xv. The electrodes are epoxied into the channels, flush to the bottom surface.

xvi. After 30 minutes, an 18 mm×8 mm piece of double sided tape is attached to the PMMA roof substrate flush to the end without channels.

xvii. The roof substrate is attached to the porous membrane.

xviii. The four edges between roof and bottom PMMA substrates are sealed with epoxy.

xix. Two 25 mm sections of tubing are cut and epoxied into the two holes in the roof substrate.

### Example 3: Binding Assay for Aggregate Building

i. Steps in binding assay may be run under flow or static conditions and have been optimized for greatest viral capture and aggregate formation when used to quantify HIV. Times may be further optimized in the future to increase signal or validate capture with other analytes.

ii. The device is functionalized with 100 μL of 10 μg/mL neutravidin (or other antibody or molecule capable of capturing a biomolecule of interest such as anti-gp120) at a flow rate of 5 μL/min or injected statically for 20 min. A static incubation at 4° Celsius for at least 2 hours can be used.

iii. 200 μL of solution containing the biomolecule of interest (e.g., HIV particles) is flowed through device at a rate of 15 μL/min under flow or injected statically for 8 minutes. Alternately, 250 uL of solution containing the biomolecule of interest (e.g., HIV) is flowed through device at a rate of 15 μL/min under flow or injected statically for 17 minutes.

iv. 100 μL of deionized water is flushed through device at a rate of 25 μL/min. This step is optional, but was used in the example described herein.

v. Gold nanoparticles are passively adsorbed to anti-gp120 antibody (or another suitable binding molecule) at a concentration of 100 μg/mL for 20 minutes at 25° Celsius, now referred to as functionalized gold nanoparticles.

vi. Functionalized gold nanoparticles are blocked with a 150 μg/mL solution of bovine serum albumin (BSA). Alternately, functionalized gold nanoparticles are blocked with a 150 μg/mL solution of bovine serum albumin (BSA) 20 minutes prior to use in the binding assay.

vii. Functionalized gold nanoparticles are resuspended in deionized water.

viii. 200 μL of functionalized gold nanoparticles is flowed through the device at a rate of 15 μL/min under flow for 8 minutes. Alternately, 250 μL of functionalized gold nanoparticles is flushed into the device using a syringe and incubated statically for 17 minutes.

ix. Component A and component B of the Sigma-Aldrich Silver Enhancer Kit are mixed in a one to one ratio.

x. 150 μL of the mixture of component A and component B are flowed through the device at a rate of 15 μL/min for 10 minutes. Alternately, 250 μL of the mixture of component A and component B is flushed into the device using a syringe and incubated statically for 17 minutes.

xi. Optionally, 75 μL of 2.5% sodium thiosulfate is flowed through device at a rate of 25 μL/min for 3 minutes. Alternately, 250 μL of 2.5% sodium thiosulfate is flushed into the devices using a syringe and incubated statically for 3 minutes.

xii. 250 μL of 1 mM K[Fe(CN)₆] in 0.1 M NaNO₃ is injected into the device.

xiii. Counter and working leads of a potentiostat are attached to the electrodes, and voltage is swept cyclically from −0.4 to +0.3 V at a rate of 33 mV/s with a step potential of 10 mV, and corresponding currents are recorded. Voltage values may be further optimized.

xiv. Peak current is identified as the highest current value in the voltage sweep, the area within the voltage-current curve is calculated, and the area and current are compared to standard curves relating their values to a viral load.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A microfluidic capture device, comprising:
a regular macroporous layer comprising a binding molecule,
an inlet and outlet port fluidly connected to the regular macroporous layer and configured to add or remove a biological sample to the regular macroporous layer and positioned to allow flow of the biological sample through a flow region in the regular macroporous layer,
a first and second electrode positioned on opposite sides of the flow region, and
a substrate enclosing the regular macroporous layer, including top and bottom sides on opposite sides of the regular macroporous layer.

2. The device of claim 1, wherein the binding molecule is an antibody.

3. The device of claim 2, wherein the antibody specifically binds to human immunodeficiency virus.

4. The device of claim 1, wherein the macroporous layer comprises polystyrene.

5. The device of claim 1, wherein the regular macroporous layer comprises pores having a diameter from about 0.5 μM to 10 μM.

6. The device of claim 1, wherein the regular macroporous layer comprises a regular array of microspherical voids.

7. The device of claim 1, wherein the substrate comprises polymethyl methacrylate.

8. The device of claim 1, further comprising an adhesive layer between the regular macroporous layer and the top and bottom sides of the substrate.

9. The device of claim 1, wherein the first and second electrodes are connected to a potentiostat.

10. The device of claim 1, wherein the top side of the substrate includes openings configured to retain the first and second electrodes.

11. The device of claim 1, wherein the top side of the substrate includes two openings that function as the inlet and outlet ports.

12. A method of detecting a biological analyte in a subject, comprising obtaining a biological sample from a subject, passing the biological sample through the flow region of a microfluidic capture device according to claim 1, passing a redox solution through the flow region of the microfluidic capture device, applying a cyclic voltage to the first electrode of the microfluidic capture device, measuring the current flow and/or area of the voltage-current curve through the microfluidic capture device, and comparing the current flow and/or voltage-current curve to a corresponding control value to determine if the biological analyte is present in the biological sample, wherein the binding molecule of the microfluidic capture device specifically binds to the biological analyte being evaluated.

13. The method of claim 12, further comprising the step of functionalizing the binding molecule with gold nanoparticles after passing the biological sample through the flow region of the microfluidic capture device.

14. The method of claim 13, further comprising depositing silver on the gold nanoparticles.

15. The method of claim 12, wherein the biological analyte is a virus particle.

16. The method of claim 15, wherein the virus particle is a human immunodeficiency virus particle.

17. The method of claim 15, wherein the subject has been diagnosed as having a viral infection, and the method is used to determine the viral load of the subject.

18. The method of claim 12, wherein the redox solution is a K[Fe(CN)₆] solution.

19. The method of claim 12, wherein the regular macroporous layer comprises a regular array of microspherical voids having a diameter of about 0.5 μM to about 10 μM.

20. The method of claim 12, wherein the subject is a human subject.
